(19) United States
(12) Patent Application Publication
(10) Pub. No.: US 2021/0350929 A1
(43) Pub. Date: Nov. 11, 2021

(54) VISUAL IDENTIFICATION BASED ACCESS CONTROL OF A PERSON WITH SUSPECTED SYMPTOMS

(71) Applicant: CORSIGHT.AI, Tel Aviv (IL)

(72) Inventor: Ofer Ronen, Tel Aviv (IL)

(73) Assignee: CORSIGHT.AI, Tel Aviv (IL)

(21) Appl. No.: 17/302,700

(22) Filed: May 11, 2021

Related U.S. Application Data
(60) Provisional application No. 63/023,207, filed on May 11, 2020.

(51) Int. Cl. G16H 50/20; G16H 50/80; G06K 9/00; G16H 10/60

(52) U.S. Cl. CPC G16H 50/20; G16H 10/60; G06K 9/00221; G16H 50/80

(57) ABSTRACT

Systems, and method and computer readable media that store instructions for thermal screening.

### BACKGROUND

One of the common symptoms of CORVID-19 is fever. In order to attempt to reduce the spreading of the CORVID-19, temperature screening has become very popular—and in some locations it is mandatory. Non-limiting examples of locations that apply temperature screening include airports, border crossings, public transportation, restaurants, hospitals, stores, malls, workplaces, and the like.

The temperature screening may be executed by thermal sensor of various types—including thermal cameras, thermometers, and the like.

People that were detected with fever may try to bypass the thermal screening in various manners. For example—attempt to pass through one or more entrances of a building in which thermal screening is not applied, prefer visiting alternative locations that are not thermally screened, and even consume a fever reduction medicine after previously detected with fever.

Furthermore—the thermal screening—especially human based thermal screening may be applied in a sub-optimal manner.

In addition—thermal screening may be applied only in a sub-set of the locations that may benefit from the thermal screening.

There is a growing need to improve thermal screening.

### SUMMARY

There may be provided systems, methods and computer readable medium as illustrated in the specification.

### BRIEF DESCRIPTION OF THE DRAWINGS

The embodiments of the disclosure will be understood and appreciated more fully from the following detailed description, taken in conjunction with the drawings in which:

FIG. 1 illustrates an example of a method;

FIG. 2 illustrates an example of a system;

FIG. 3 illustrates an example of an asset and thermal screening; and

FIG. 4 illustrates an example of an asset and thermal screening.

### DESCRIPTION OF EXAMPLE EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a device or system capable of executing the method and/or to a non-transitory computer readable medium that stores instructions for executing the method.

Any reference in the specification to a system or device should be applied mutatis mutandis to a method that may be executed by the system, and/or may be applied mutatis mutandis to non-transitory computer readable medium that stores instructions executable by the system.

Any reference in the specification to a non-transitory computer readable medium should be applied mutatis mutandis to a device or system capable of executing instructions stored in the non-transitory computer readable medium and/or may be applied mutatis mutandis to a method for executing the instructions.

Any combination of any module or unit listed in any of the figures, any part of the specification and/or any claims may be provided.

The specification and/or drawings may refer to an image. An image is an example of a media unit. Any reference to an image may be applied mutatis mutandis to a media unit. A media unit may be an example of sensed information unit. Any reference to a media unit may be applied mutatis mutandis to sensed information. The sensed information may be sensed by any type of sensors—such as a visual light camera, or a sensor that may sense infrared, radar imagery, ultrasound, electro-optics, radiography, LIDAR (light detection and ranging), etc.

The specification and/or drawings may refer to a processor. The processor may be a processing circuitry. The processing circuitry may be implemented as a central processing unit (CPU), and/or one or more other integrated circuits such as application-specific integrated circuits (ASICs), field programmable gate arrays (FPGAs), full-custom integrated circuits, etc., or a combination of such integrated circuits.

Any combination of any steps of any method illustrated in the specification and/or drawings may be provided.

Any combination of any subject matter of any of claims may be provided.

Any combinations of systems, units, components, processors, sensors, illustrated in the specification and/or drawings may be provided.

The analysis of content of a media unit may be executed by generating a signature of the media unit and by comparing the signature to reference signatures. The reference signatures may be arranged in one or more concept structures or may be arranged in any other manner. The signatures may be used for object detection or for any other use.

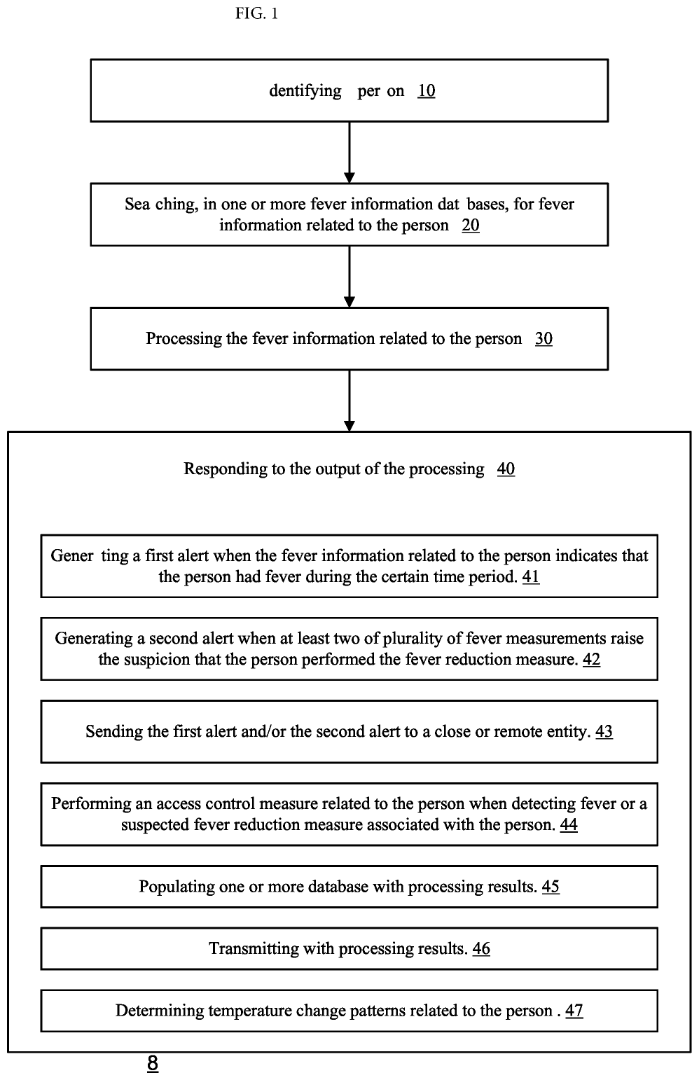

FIG. 1 illustrates method 8 for thermal screening.

Method 8 may start by step 10 of identifying a person.

The person is identified as being located at a certain location. The certain location may be anywhere—especially a location in which thermal sensors are positioned. For example—the certain location may be an asset that may condition access by thermal screening. The thermal screening may occur at an entrance or any other location within a building or near a building, an entrance or any other location within a public transportation facility or vehicle or near a public transportation facility or vehicle, an entry point of any other point within or near a public area, a national park, a playground, and the like. A public transportation facility may be an airport, a bus station, a train station, a taxi station, a maritime facility such as a port. A vehicle may be an airborne vehicle, a maritime vehicle and/or a ground vehicle. The building may be any building—for example a museum, a building store, a mall, and the like.

The thermal sensors may be contact-based thermal sensors, contactless thermal sensors and the like. Non-limiting example of thermal sensors may include thermal cameras, thermometers, and the like.

The identifying of the person may be executed with or without human intervention. For example—the human may provide an identifying document. Yet for another example—the person may be identified by one or more sensors—for example an image sensor, by a thermal sensor that provides a thermal image—such as a thermal camera, and the like.

Any one or more images of the person may be processed in various manners—by applying a deep neural network, by applying a machine learning process, by generating signatures of the images, by face recognition algorithms, and the like. An example of a signature generation process and/or object detection is illustrated in U.S. patent application Ser. No. 16/542,327 which is incorporated herein by reference.

Step 10 may be followed by step 20 of searching, in one or more fever information databases, for fever information related to the person. The searching may be executed in real time (for example with a latency that does not exceed few seconds) and may involve accessing and scanning one or many databases.

A fever information database may include only information regarding fever measurements or may include additional information—such as additional medical information.

The one or more fever information databases may be located in one or more locations, may be managed by one or more entities. The one or more fever information databases may be accessed in any manner—by a web browser, by an application, over any network, over a secure link, over an unsecure link, and the like.

The one or more fever information databases may include measurements taken at different locations, by different entities such as in medical facilities, or in any location where thermal screening is applied.

By obtaining fever information from different location the method allows to perform thermal screening at a location even without measuring the temperature of the at that location.

The fever information related to the person is indicative of an outcome of at least one fever measurement taken of the person during a certain time period that preceded the detecting of the person.

The certain time period may be the same for all persons, may differ from one person to the other, may be determined based on one or more parameters such as age, gender, medical profile, environmental conditions (rain, humidity, temperature, and the like) or may be determined in any other manner.

The certain time period may be one or more hours, one or more days, and the like.

The fever information of the person may include location and timing information about a location and timing of each fever measurement.

The one or more fever information databases may be are populated by multiple fever measurements taken at multiple locations.

The fever information related to any person may be associated with an identifier of the person. The identifier of the person may be an image signature, or any other identifier generated during step 10, or may include an identifier that differs from an image signature (for example a persons' passport number, social security number of other ID.), and the like. Step 10 of identifying of the person may include generating a detected person identifier. Step 20 may include searching for fever information associated with an identifier of a person that matches the detected person identifier.

Step 20 may be followed by step 30 of processing the fever information related to the person. This may include, for example, determining if the fever information is indicative that the person had fever during the certain time period, and/or determined whether two or measurements raise the suspicion that the person performed a fever reduction measure such as taking fever reduction medicine, cooling himself, and the like.

Step 30 may be followed by step 40 of responding to the output of the processing.

Step 40 may include at least some of the following steps:
Step 41 of generating a first alert when the fever information related to the person indicates that the person had fever during the certain time period.
Step 42 of generating a second alert when at least two of a plurality of fever measurements raise the suspicion that the person performed the fever reduction measure.
Step 43 of sending the first alert and/or the second alert to a close or remote entity such as a governmental entity or a private entity. For example sending an alert to a health department, to the police, to a person or organization which controls an access to a building or facility or any other asset which may be protected by the thermal screening, alerting vendors, service providers of people located at the vicinity of the person about that the fever or a suspected fever reduction measure associated with the person, and the like.
Step 44 of performing an access control measure related to the person when detecting fever or a suspected fever reduction measure associated with the person. The access control measure may include controlling an automatic door by not opening—or closing the automatic door to prevent the person from proceeding through the door. The access control measure may also include informing an entity which manages the access to the asset.
Step 45 of populating one or more database with processing results—for example processing results regarding the fever or a suspected fever reduction measure associated with the person.
Step 46 of transmitting with processing results—for example processing results regarding the fever or a suspected fever reduction measure associated with the person.
Step 47 of determining temperature change patterns related to the person—by taking into account the temperature measurements and the time in which the measurements were taken.

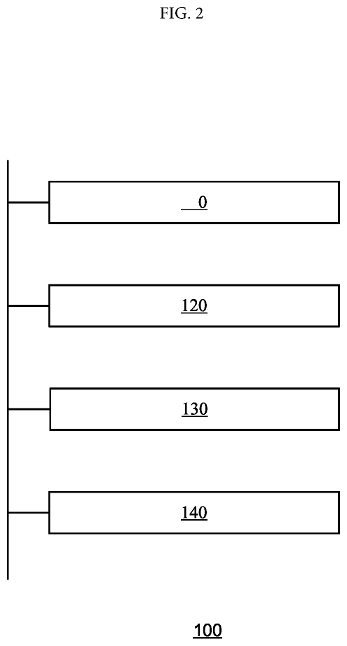

FIG. 2 illustrates an example of a computerized system 100 and various fever information databases 101.

Computerized system 100 may include a person identification unit 110, a search engine 120, a decision unit 130 and an input output unit 140.

The person identification unit 110 is configured to identify a person.

The search engine 120 is configured to search, in one or more fever information databases, for fever information related to the person. The fever information related to the person is indicative of an outcome of at least one fever measurement taken of the person during a certain time period that preceded the detecting of the person.

The decision unit 130 is configured to process the fever information related to the person and to respond to the outcome of the processing.

The decision unit 130 may be configured to execute step 30 and may be configured to execute (or at least partially execute) step 40.

Each one or person identification unit 110, search engine 120 and decision unit 130 may be a computerized unit that may include one or more processing circuit and one or more memory units.

For example—the decision unit 130 may be configured to generate a first alert when the fever information related to the person indicates that the person had fever during the certain time period.

Input output unit 140 may receive and/or output information, and/or alerts and/or reports, and the like. Input output unit 140 may be any suitable communications component such as a network interface card, universal serial bus (USB) port, disk reader, modem or transceiver that may be operative to use protocols such as are known in the art to communicate either directly, or indirectly, with other elements of system 100 and/or other entities.

Search engine 120 may access the one or more fever information databases via input output unit 140.

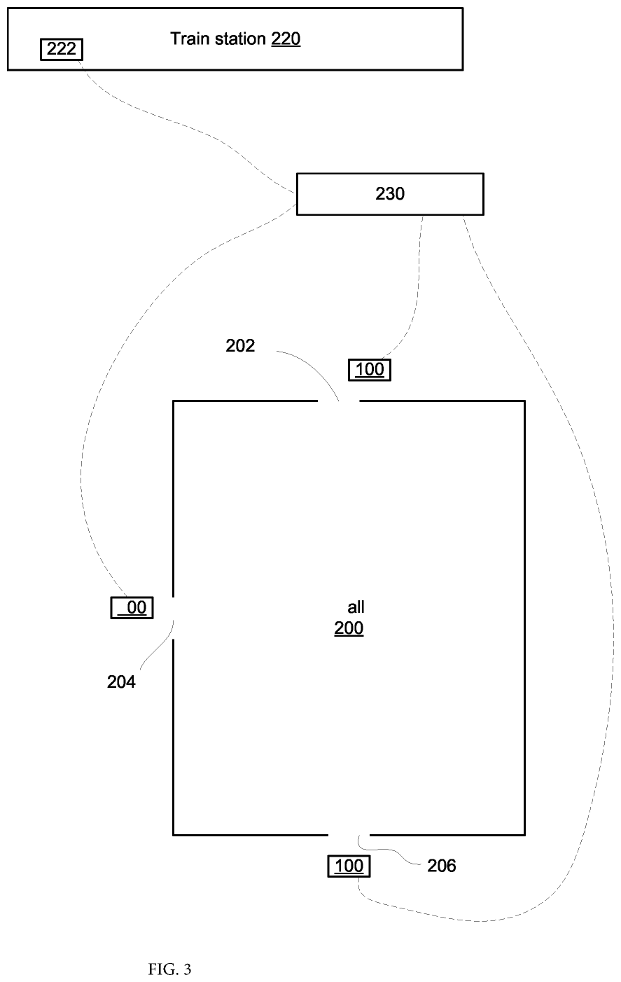

FIG. 3 illustrates an asset 200 and thermal screening.

The asset 200 is a mall that has multiple entrances—first entrance 202, second entrance 204 and third entrance 206. The mall 200 is located at the vicinity of a train station 220 that has a train station temperature measurement check point 222 for measuring the temperature of persons that enter or exit the train station 220.

Assuming, for example, that none of the entrances of the mall is equipped with temperature measurement check points—but that computerized systems 100 are located near each of the first, second and third entrances. The computerized systems 100 may access one or more fever information databases 230 that may store fever information obtained in the train station temperature measurement check point 222—or in any other locations. In this case, thermal screening can be performed near each one of the entrances—even without measuring temperatures near the entrances.

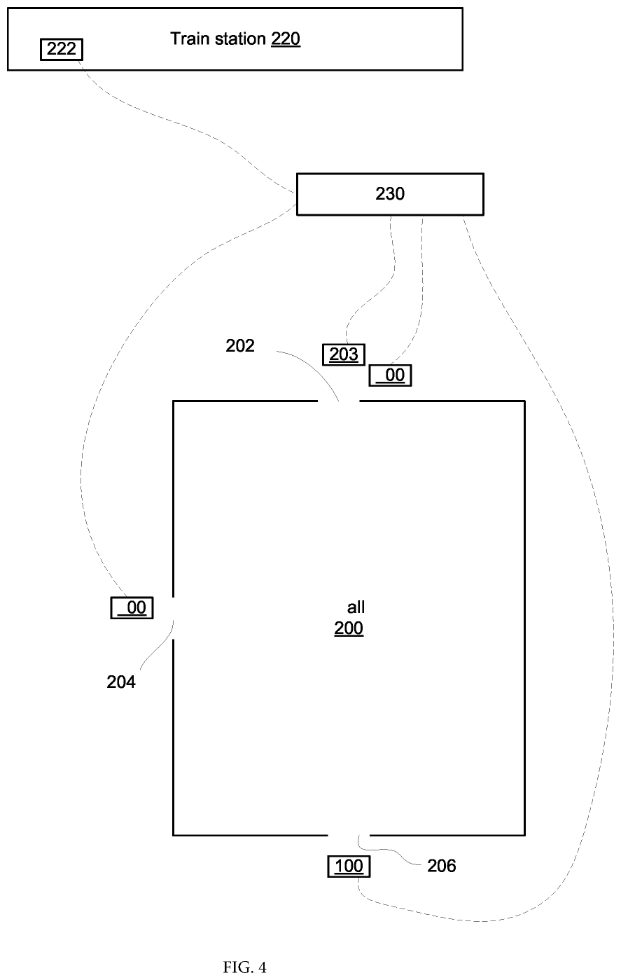

FIG. 4 illustrates an asset 200 and thermal screening.

The asset 200 is a mall that has multiple entrances—first entrance 202, second entrance 204 and third entrance 206. The mall 200 is located at the vicinity of a train station 220 that has a train station temperature measurement check point 222 for measuring the temperature of persons that enter or exit the train station 220.

There is a first mall temperature measurement check point 203 near the first entrance 202. In this case, thermal screening can be performed near each one of the second and third entrances—even without measuring temperatures near these entrances. The thermal screening may include searching fever information at one or more fever information databases 230 that may store fever information obtained in the train station temperature measurement check point 222 and/or obtained at first mall temperature measurement check point 203, and/or obtained at any other place.

It should be noted that the screening of the person may be based on a measurement other than a fever measurement—and that any reference to a thermal screening should be applied to screening based on any other sickness symptom, and that any reference to fever may be applicable to any other sickness symptom detectible by any type of detection of a symptom of sickness—for example behavior patterns based detection, skin coloring based detection, symptoms such as changes that can be detected through unique light wavelengths.

It should be noted that fever information from different locations may be compared to each other and/or may be used to verify the measurements of one or more temperature measurement check points. For example—differences between multiple measurements of the same people between one check point to another may indicate in inaccuracy in fever measurements such as offsets in the measurements.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Furthermore, the terms “assert” or “set” and “negate” (or “deassert” or “clear”) are used herein when referring to the rendering of a signal, status bit, or similar apparatus into its logically true or logically false state, respectively. If the logically true state is a logic level one, the logically false state is a logic level zero. And if the logically true state is a logic level zero, the logically false state is a logic level one.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It is appreciated that various features of the embodiments of the disclosure which are, for clarity, described in the contexts of separate embodiments may also be provided in combination in a single embodiment. Conversely, various features of the embodiments of the disclosure which are, for brevity, described in the context of a single embodiment may also be provided separately or in any suitable sub-combination.

It will be appreciated by persons skilled in the art that the embodiments of the disclosure are not limited by what has been particularly shown and described hereinabove. Rather the scope of the embodiments of the disclosure is defined by the appended claims and equivalents thereof.

What is claimed is:

1. A method for thermal screening, the method comprises:
identifying a person;
searching, in one or more fever information databases, for fever information related to the person; wherein the fever information related to the person is indicative of an outcome of at least one fever measurement taken of the person during a certain time period that preceded the detecting of the person; and
generating a first alert when the fever information related to the person indicates that the person had fever during the certain time period.

2. The method according to claim 1 wherein the identifying is executed at a certain location and wherein a fever measurement of the at least one fever measurements is taken at a location that differs from the certain location.

3. The method according to claim 1 wherein the one or more fever information databases are populated by multiple fever measurements taken at multiple locations.

4. The method according to claim 1 wherein fever information related to any person is associated with an identifier of the person; wherein the identifying of the person comprises generating a detected person identifier; wherein the searching comprises searching for fever information associated with an identifier of a person that matches the detected person identifier.

5. The method according to claim 1 wherein the fever information related to the person is indicative of outcomes of a plurality of fever measurements taken of the person during the certain time period.

6. The method according to claim 5 comprising determining whether at least two of the plurality of fever measurements raise a suspicion that the person performed a fever reduction measure.

7. The method according to claim 6 comprising generating a second alert when the at least two of the plurality of fever measurements raise the suspicion that the person performed the fever reduction measure.

8. The method according to claim 1 wherein fever information of the person comprises location and timing information about a location and timing of each fever measurement.

9. The method according to claim 1 comprising closing or not opening an automatic door when the fever information of the person indicates that the person had fever during the certain time period.

10. The method according to claim 1 wherein the identifying comprising acquiring one or more images and applying a face recognition process on the one or more images.

11. The method according to claim 1 wherein the identifying comprising acquiring one or more images and applying a machine learning process to identify the person in the one or more images.

12. The method according to claim 1 comprising sending the first alert to a governmental entity.

13. A non-transitory computer readable medium for thermal screening, the non-transitory computer readable medium stores instructions for:
identifying a person;
searching, in one or more fever information databases, for fever information related to the person; wherein the fever information related to the person is indicative of an outcome of at least one fever measurement taken of the person during a certain time period that preceded the detecting of the person; and
generating a first alert when the fever information related to the person indicates that the person had fever during the certain time period.

14. The non-transitory computer readable medium according to claim 13 wherein the identifying is executed at a certain location and wherein a fever measurement of the at least one fever measurements is taken at a location that differs from the certain location.

15. The non-transitory computer readable medium according to claim 13 wherein the one or more fever information databases are populated by multiple fever measurements taken at multiple locations.

16. The non-transitory computer readable medium according to claim 13 wherein fever information related to any person is associated with an identifier of the person;
wherein the identifying of the person comprises generating a detected person identifier; wherein the searching comprises searching for fever information associated with an identifier of a person that matches the detected person identifier.

17. The non-transitory computer readable medium according to claim 13 wherein the fever information related to the person is indicative of outcomes of a plurality of fever measurements taken of the person during the certain time period.

18. The non-transitory computer readable medium according to claim 17 that stores instructions for determining whether at least two of the plurality of fever measurements raise a suspicion that the person performed a fever reduction measure.

19. The non-transitory computer readable medium according to claim 18 that stores instructions for generating a second alert when the at least two of the plurality of fever measurements raise the suspicion that the person performed the fever reduction measure.

20. A computerized system that comprises a person identification unit, a search engine and a decision unit, wherein the person identification unit is configured to identify a person; wherein the search engine is configured to search, in one or more fever information databases, for fever information related to the person; wherein the fever information related to the person is indicative of an outcome of at least one fever measurement taken of the person during a certain time period that preceded the detecting of the person; and wherein the decision unit is configured to generate a first alert when the fever information related to the person indicates that the person had fever during the certain time period.

21. A method for screening, the method comprises:
identifying a person;
searching, in one or more sickness symptom information databases, for sickness symptom information related to the person; wherein the sickness symptom information related to the person is indicative of an outcome of at least one sickness symptom measurement taken of the person during a certain time period that preceded the detecting of the person; and
generating a first alert when the sickness symptom information related to the person indicates that the person exhibited a sickness symptom during the certain time period.

22. A non-transitory computer readable medium for thermal screening, the non-transitory computer readable medium stores instructions for:
identifying a person;
searching, in one or more sickness symptom information databases, for sickness symptom information related to the person; wherein the sickness symptom information related to the person is indicative of an outcome of at least one sickness symptom measurement taken of the person during a certain time period that preceded the detecting of the person; and
generating a first alert when the sickness symptom information related to the person indicates that the person exhibited a sickness symptom during the certain time period.

23. A computerized system that comprises a person identification unit, a search engine and a decision unit, wherein the person identification unit is configured to identify a person; wherein the search engine is configured to search, in one or more sickness symptom information databases, for sickness symptom information related to the person; wherein the sickness symptom information related to the person is indicative of an outcome of at least one sickness symptom measurement taken of the person during a certain time period that preceded the detecting of the person; and wherein the decision unit is configured to generate a first alert when the sickness symptom information related to the person indicates that the person exhibited sickness symptom during the certain time period.
